(19) United States
(12) Patent Application Publication
(10) Pub. No.: US 2017/0027858 A1
(43) Pub. Date: Feb. 2, 2017

(54) DEVICE AND METHOD TO CONTROL RELEASE OF COMPOUND

(71) Applicant: PURDUE RESEARCH FOUNDATION, West Lafayette, IN (US)

(72) Inventors: Richard B. BORGENS, Delphi, IN (US); John CIRILLO, Oxford, IN (US); Wen GAO, Lafayette, IN (US)

(73) Assignee: Purdue Research Foundation, West Lafayette, IN (US)

(21) Appl. No.: 15/301,042

(22) PCT Filed: Mar. 31, 2015
(86) PCT No.: PCT/US15/23444

§ 371 Date: Sep. 30, 2016

Related U.S. Application Data
(60) Provisional application No. 61/972,991, filed on Mar. 31, 2014.

(51) Int. Cl. A61K 9/00; A61K 9/16

(52) U.S. Cl. CPC A61K 9/0009; A61K 9/0024; A61K 9/1611; A61K 9/1635

(57) ABSTRACT

An implantable time-release delivery system is disclosed, comprising at least one nano-complex of a plurality of vertically aligned rods fixed at one end to a substrate and configured to be implantable within a body, the plurality of vertically aligned rods comprising polypyrrole, gold nanoparticles, and a compound. An electromagnetic field generating device configured to generate an electromagnetic field, positioned in a near field arrangement with respect to the nano-complex, the electromagnetic field causes release of the compound from the nano-complex into the body.

### CROSS-REFERENCE TO RELATED APPLICATIONS

The present U.S. patent application is related to and claims the priority benefit of U.S. Provisional Patent Application Ser. No. 61/972,991, filed Mar. 31, 2014, the contents of which is hereby incorporated by reference in its entirety into this disclosure.

### TECHNICAL FIELD

The present disclosure generally relates to the controlled release of a compound by a stimulus, and in particular to a device and method that uses conductive polymers and materials to hold and release a compound when stimulated by electromagnetic fields.

### BACKGROUND

This section introduces aspects that may help facilitate a better understanding of the disclosure. Accordingly, these statements are to be read in this light and are not to be understood as admissions about what is or is not prior art.

There are growing clinical demands for controlled and sustained drug release systems to serve as implantable devices for patients with acute and chronic diseases. Under these circumstances, it is not surprising that intelligent materials have emerged as a promising strategy for drug delivery. For example, many efforts have been directed toward using various stimuli-responsive biomaterials as “on-off” controllable drug carriers in which the bioactive cargos are released via changes in pH, temperature or input of electrical or UV energy. At the present time, electrical stimulation appears to be one of the more suitable approaches for clinical translation in that: 1) the electrical signal can be triggered using portable equipment, not requiring significant cost or sophisticated technologies; and 2) the generated signal can be tuned using a variety of means or devices.

In terms of stimuli-responsive biomaterials, polypyrrole (Ppy) in particular has become a candidate material due to its lack of toxicity, favorable biocompatibility, and reversible electrochemical properties. The electrostatic interaction of Ppy in response to electric current provides a controllable “switch” for the release of tethered cargo, providing in situ delivery of nerve growth factors, pain-relief drugs, or chemotherapeutic compounds. Prior investigations demonstrate time and site-specific release profiles can be obtained by modifying electrical pulse patterns and durations.

However, two obstacles that prevent the practical use of Ppy polymer in medical systems are: 1) the amount of a drug cargo is limited when using typical flat thin film fabrication, and 2) delivery of the cargo within the human body requires percutaneous electrodes to deliver the required level of electric current (i.e. a physical electrical contact to the Ppy substrate). This latter obstacle must be understood in the context of chronic applications where drug release may be desirable over many days until the supply within the Ppy substrate is exhausted. During this time, percutaneous wires carry the possibility of infection by retrograde tracking along the insertion path where normal movement disrupts a perfect seal between tissues and the insulated electrodes.

### SUMMARY

According to one aspect, an implantable time-release delivery system is disclosed, comprising a nano-complex of a plurality of vertically aligned rods fixed at one end to a substrate and configured to be implantable within a body, the plurality of vertically aligned rods comprising polypyrrole, gold nanoparticles, and a compound. An electromagnetic field generating device is configured to generate an electromagnetic field when positioned in a near field arrangement with respect to the nano-complex. The electromagnetic field causes release of the compound from the nano-complex into the body.

According to another aspect, a method of controlling compound release is disclosed, comprising providing a nano-complex of a plurality of vertically aligned rods fixed at one end to a substrate and configured to respond to an electromagnetic field, the plurality of vertically aligned rods comprising polypyrrole, gold, and at least one compound, providing a pulse of electromagnetic energy to the nano-complex in a range of 20 to 40 G, to thereby release the at least one compound from the polypyrrole complex, and stopping the pulse of electromagnetic energy to thereby stop the release of the at least one compound.

### BRIEF DESCRIPTION OF THE DRAWINGS

FIG. 1 is an illustrative example of a nano-complex of a plurality of vertically aligned rods fixed at a proximal end to a substrate, the plurality of vertically aligned rods comprising a conducting polymer, gold, and a compound.

FIG. 2 illustrates a process for forming the nano-complex of FIG. 1.

FIG. 3 shows an illustration of an electromagnetic field generating device configured to generate an electromagnetic field in a continuous or pulsing waveform to control release of a compound in the nano-complex of FIG. 1 when implanted in a test subject.

FIGS. 4a and 4b shows an input signal used to energize a coil of the device of FIG. 3 according to one embodiment.

FIGS. 5a and 5b show an SEM micrograph of the nano-complex of FIG. 1.

FIG. 6 shows DEX release test results using the nano-complex of FIG. 1.

FIG. 7 shows bioluminescence of GFAP in test animal subjects according to one embodiment.

FIG. 8 shows differences in GFAP intensity as a function of time post injury in test animal subjects of FIG. 7.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure has been made in an effort to provide a nano-complex capable of controlling the release of at least one compound by the presence and absence of electromagnetic energy, a method of manufacturing the nano-complex, and a method and apparatus for controlling release of the at least one compound from the nano-complex.

An exemplary embodiment of the disclosure provides a nano-complex of a plurality of vertically aligned rods fixed at one end to a substrate and configured to respond to an electromagnetic field, the plurality of vertically aligned rods comprising a conducting polymer, gold, and a compound; and an electromagnetic field generating device configured to generate the electromagnetic field, positioned in a near field arrangement with respect to the nano-complex, the electromagnetic field causes release of the compound from the nano-complex.

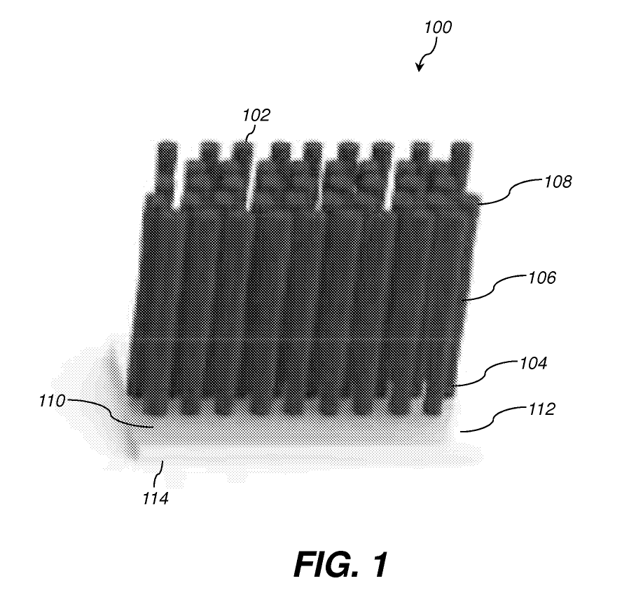

Referring now to FIG. 1, the nano-complex 100 is a three dimensional structure comprising a plurality of vertically aligned rods 102. The vertically aligned rods 102 are distinct structures with space between each aligned rod 102. Each aligned rod 102 comprises three portions, a proximal end 104, a middle portion 106, and a distal end 108. At one end of the nano-complex 100, each proximal end 104 of the aligned rods 102 is coupled to a substrate 110. At least the distal end 108 of each aligned rod 102 is exposed to the environment.

The substrate 110 coupled to the proximal end 104 of the aligned rods 102 includes an attachment member 112 and an attachment edge 114. The aligned rods 102 couple to the attachment member 112 of the substrate 110. Each substrate 110 may have a first attachment member and a second attachment member. The attachment edge 114 is used to couple other attachment edges 114 together to generate a complex comprising more than one nano-complex 100. Several substrates 110 may be coupled together to form a shape similar to a cube, a triangle, a polyhedron, or a sphere-like shape. The substrate 110 may be made of various materials with an exemplary material being gold.

The nano-complex 100 may include a conducting polymer, gold nanoparticles, a compound, or combinations thereof. The conducting polymer is formed into vertically aligned rods 102 of a pre-specified height and thickness. The vertically aligned rods 102 may range in height from 50 nano-meters (nm) to 15000 nm. The vertically aligned rods 102 may range in a thickness of 20 nm to 2000 nm. The gold nanoparticles may be coupled to the aligned rods 102. The compound may be coupled to the aligned rods 102 in a manner that allows release of the compound once the conducting polymer is reduced when stimulated.

By “conducting polymer” means a polymer having high conductivity. A few examples of a conducting polymer are polypyrrole or polyacetylene.

By “compound” means a molecule which will be released into the surrounding environment. Examples of what is intended to be meant by compound is a pharmaceutical drug, an imaging agent, a peptide, a metabolite, a vitamin, a small molecule, a eukaryotic cell, a stem cell, a fatty-acid, a cholesterol, a steroid, a toxin, a prodrug, a pharmaceutical acceptable salt, a salt with deliverable ions, metallic particles, crystalline particles, an enzyme, a ligand, a synthetic peptide, a DNA molecule, a cDNA molecule, an RNA molecule, a prokaryotic cell, a virus, a chemotherapeutic, or a radiotherapeutic.

The amount of compound that can be coupled to the nano-complex 100 will depend on the size of the compound, the height of the nano-complex 100, and the width of the nano-complex 100.

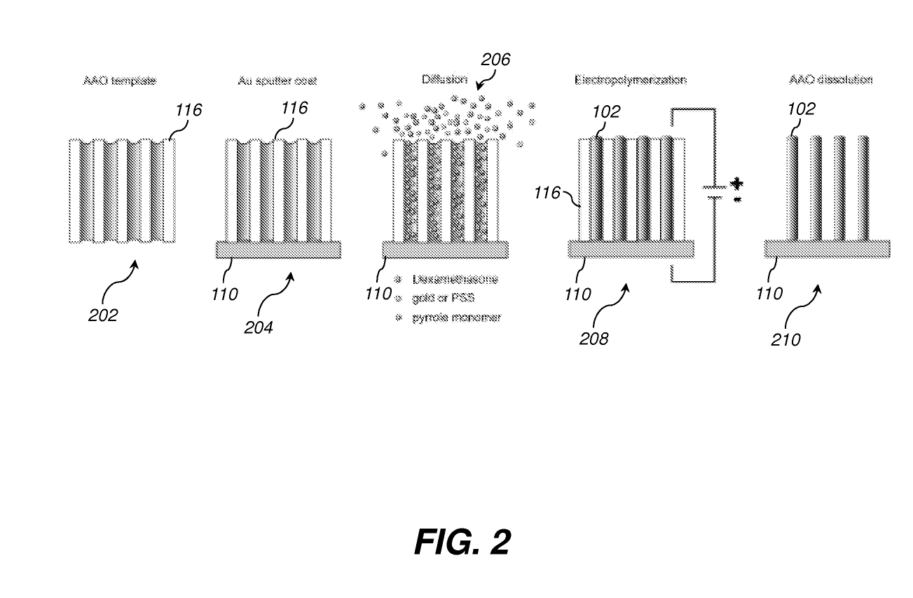

FIG. 2 illustrates a process for fabricating the nano-complex 100. First, the AAO template 116 is prepared (202). Next, the template is coated with gold on one side (204). Next, the solution containing polypyrole, gold nanoparticles, and the compound is filled and diffused into the template pores (206). Next, the rods 102 are formed by applying a voltage to the template to cause electropolymerization (208). Lastly, the template 116 is dissolved (e.g., by sodium hydroxide) to leave the rods 102 and substrate 110 (step 210).

The following is an exemplary method to produce the nano-complex having vertically aligned rods. An aluminum oxide (AAO) template is provided having pores the length of the template to generate the individual vertically aligned rods. To generate the aligned rods using polypyrrole, a 100 nm thick layer of gold was deposited by a Varian E-beam Evaporator onto one side of the Anodic aluminum oxide (AAO) template. AAO templates with 0.2 micrometer pore size and 60 micrometer thickness (Whatman) were used in the fabrication process. All templates were stored in a dry oven for use.

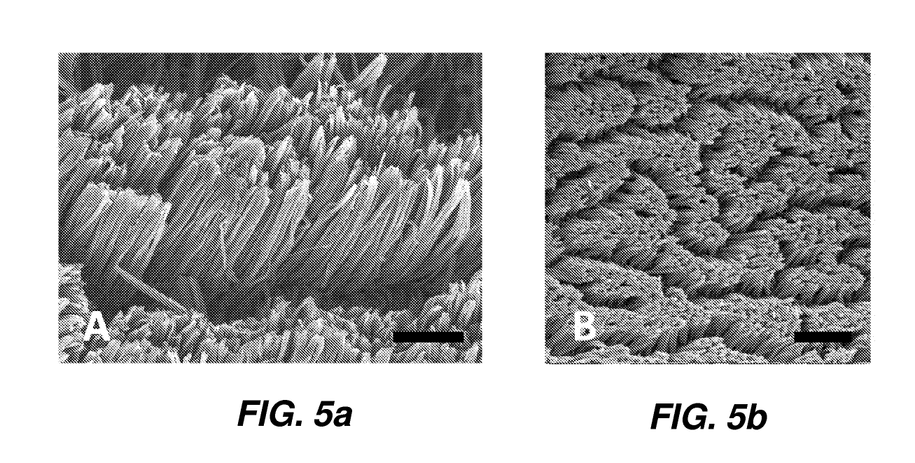

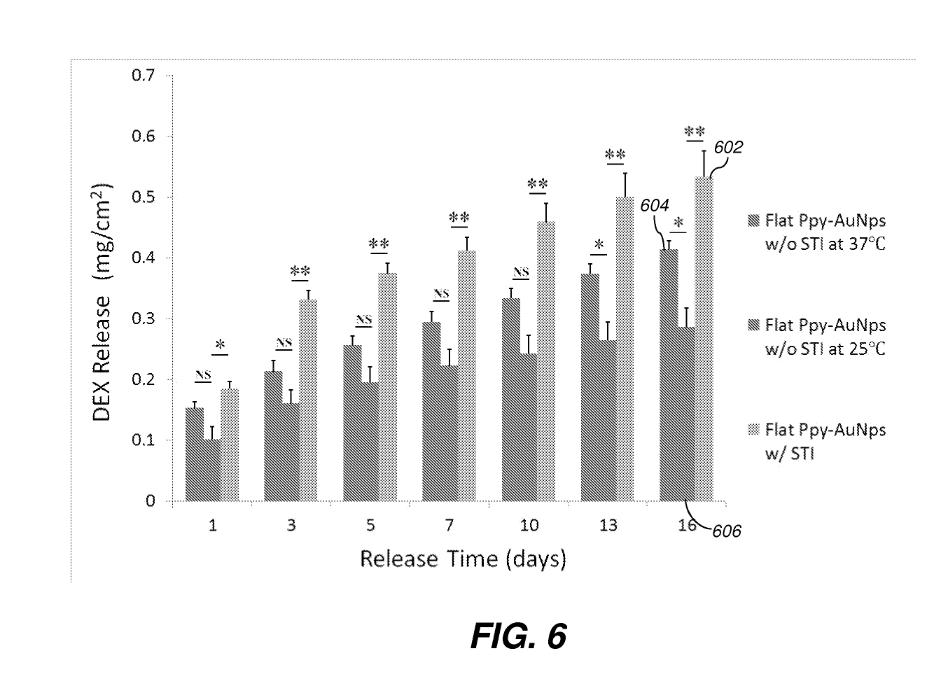

The following is an exemplary method to produce a nano-complex comprising polypyrrole, gold nanoparticles, and a pharmaceutical compound, dexamethasone 21-phophate disodium salt (DEX). 0.2M pyrrole, 0.025M DEX, 0.05 M 10 nanometer NANOXACT SPHERICAL GOLD NANOPARTICLES were mixed for synthesis. Pre-prepared AAO templates were incubated into the synthesis solution for 30 minutes. AAO templates were connected to the working electrode of the CH INSTRUMENT MODEL 604 electrochemical analyzer/workstation, with a platinum counter electrode and Ag/AgCl reference electrode in the synthesis solution. The electropolymerization of DEX/Ppy was accomplished by applying a constant potential of 1 V using a potentiostat. Then the films were rinsed thoroughly with Milli-Q water for five minutes and dried. The AAO templates were removed by placing the films in 3M sodium hydroxide for approximately eight minutes, and then rinsed with Milli-Q water. The length of grown vertically aligned rods is dependent on polymerization time. 10 micrometer long polypyrrole aligned rods were completed in about 1300-1400 seconds of deposition time using the potentiostat, while shorter lengths were realized for faster deposition times. As another example, 200 nm long vertically aligned rods can be fabricated using an AAO template with 0.02 micrometer pores. The resulting nano-complexes vary in width depending on the width of the template. The nano-complexes may be coupled together to form a larger complex in a variety of shapes, or the nano-complex may be cut to produce a smaller plurality of nano-complexes from a single template. FIGS. 5a and 5b SEM micrographs of nano-complexes produced using the above method. FIG. 6 shows further test results of the DEX release from such nano-complexes with EMF stimulation (602), compared to DEX release without stimulation at 37 degrees C. (604) and 25 degrees C. (606).

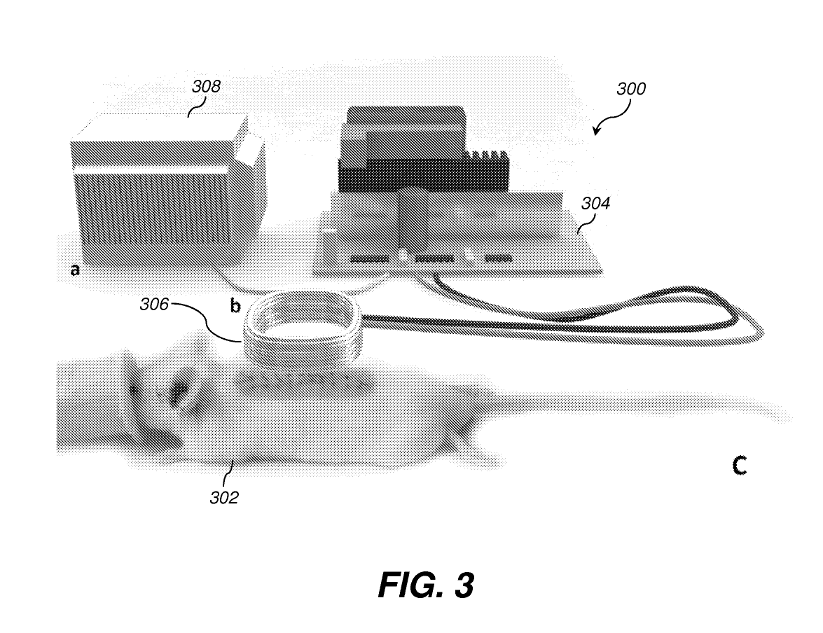

An electromagnetic field generating device 300 configured to generate the electromagnetic field is shown in FIG. 3. The device 300 is configured to generate an electromagnetic field in a continuous or pulsing waveform to control release of a compound in the nano-complex 100 implanted in a test subject, such as a mouse 302. The electromagnetic field generating device 300 comprises an electromagnetic generator 304, a coil 306, and a power source 308. It shall be understood that additional components required to produce the pulse waveform may be included in the device 300 depending on the needs of the particular application. For example, the device 300 may include computer processors, memory, and storage devices capable of storing and executing computer readable instructions for controlling the device 300 and the nano-complex 100.

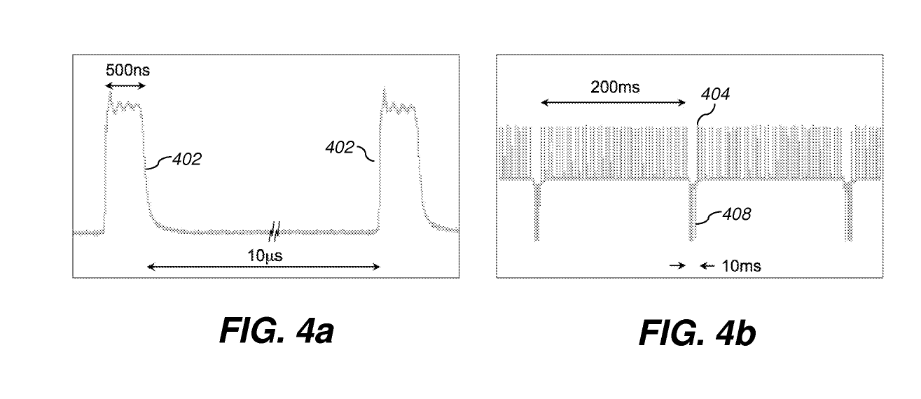

The nano-complex 100 is coupled to the electromagnetic field generating device 300 to receive stimulation. When the nano-complex 100 receives stimulation from the electromagnetic field generating device 300, the aligned rods 102 physically change. This physical change allows a coupled compound to release from the nano-complex 100. Once the stimulation is no longer present, the aligned rods 102 revert back to the original structure, and compound is no longer able to leave the nano-complex 100. The electromagnetic field generating device 300 pulses EMF directed towards the nano-complex 100 implanted in the subject 302. In one embodiment, the pulses may last between between 200 and 700 nano-seconds (nS), although other pulse durations may be used. FIG. 4a shows example pulses 402. FIG. 4b shows an embodiment where a plurality of positive pulses 404 are applied in succession, after which at least one negative pulse 408 is applied.

The following is an exemplary method of generating the electromagnetic field generating device 300. Multiple factors must be considered when designing the EMF stimulation system and pulse conditions. These parameters include waveform shape, pulse duration, pulse magnitude, duty cycle, etc. We originally chose a narrow pulse width (500 nS, as shown in FIG. 4a) stimulating regime as it allowed us to experiment with various coil geometries with minimal power consumption. The square wave was chosen in order to maximize the induced electric fields within the Ppy since that is the hypothesized stimulus for drug release. Our original pilot measurements were made using a three-tum coil which had a very low inductance allowing testing that set the limits for overheating. This data (not shown) then permitted the construction of a 15 tum coil. The geometry of the coil was 2.3×2.8 cm inside dimensions and 3.0×3.8 cm outside dimensions. Each input pulse was only “on” long enough to saturate the coil, and then turned off for the duration required to completely unload the coil. Therefore, the duty cycle was approximately 4.8%. Additionally, several stimulation patterns were tested such as oscillating the polarity, grouping same polarity pulses in different temporal patterns, etc. while maintaining about 5% duty cycle. Pulses of 500 ns duration were spaced 10 μs apart. As shown in FIG. 5b, these pulses 404 were in the positive direction for 200 ms and then alternated to the reverse polarity (shown as negative pulses 408) for 10 ms.

The following is an exemplary method of measuring the EMF produced by the electromagnetic field generating device. Using the waveform patterns described above as the input signal, the real time magnetic and electric field output from the stimulation coil were measured using high frequency EMC 100B (magnetic) and 100C (electric) probes. Probes were connected to a TEKTRONICS TDS 2012B oscilloscope and terminated with a 50 ohm resistor. For data collection, each probe was positioned in the stimulation coil center, origin. The probes were directional and oriented in the direction of the maximum expected reading. This value was taken to be the output probe value and converted to dBm. The recorded data points from the oscilloscope were imported into MS Excel in which the Fast Fourier Transform (FFT) of the waveforms was calculated. From the FFTs, the fundamental frequency, along with the output dBm power value were used with the antenna gain equation provided for each probe to estimate AC field magnitudes.

The following is an exemplary method of stimulating the nano-complex to cause release of a pharmaceutical compound. In this instant case, the pharmaceutical compound is DEX, described earlier. Pulsed electromagnetic field (EMF) is used to release DEX. The DEX/Ppy nano-complexes were stimulated by an induced EMF generated by our electromagnetic field generating device. Experiments comparing the DEX conjugated polypyrrole constructions were continued for up to 16 days. To test if the stimulated release of DEX might be influenced by the heating of stimulated samples. For all of the 16 days' DEX release, the sample solutions were collected at 1, 3, 5, 7, 10, 13, and 16 days of continuous EMF stimulation (n=5). To determine the characteristics of pulsatile EMF stimulation, “on and off” experiments were performed on polypyrrole vertically aligned rods coupled with gold nanoparticles. The DEX release profile recordings were begun after 10 hours of stimulation by the pulsed EMF. Then, pulsed EMF stimulation was turned on (referred to as “on” time) for 2 hours and discontinued (“off” time) for 2 hours. This regimen was continued for four cycles for each sample. The electromagnetic field generating device may cause stimulation activity from the nano-complex at a distance range of about 0.001 cm to 3 cm.

### EXAMPLES

The nano-complexes comprising Ppy, gold nanoparticles, and DEX were placed in a cuvette filled with a buffer solution. The cuvette was positioned in such a manner as to prevent any physical contact with the stimulation coil. The coil was subsequently energized using square wave pulse trains. The measurements revealed that the magnetic field output was similar to the input field, with some oscillation noise present in the square waveform. FFT decomposition of the measured signal showed the fundamental frequency of the magnetic field was to be 3.2 MHz. This converts to average peak amplitude of 36 Gauss when using the antenna gain equation supplied by the probe vendor. Moreover, the magnetic field as a function of distance along the z axis (height) did not vary by more than 20% within the coil. Therefore, based on these values, the nano-complexes were exposed to peak magnetic fields within the range of 25-40 Gauss. The electric field waveform exhibited oscillatory behavior with sharp peaks primarily concentrated at the “ramp-up” and “switch off” phases of the EMF. The 15-turn coil produced a peak E-field of 4700V/m and a fundamental frequency of 65 MHz. Thus, the estimated electric field magnitude in which the Ppy nano-complexes resided is in the 3000-5000 V/m range for the 15-turn coils. We emphasize that due to the high frequency pulsed nature of the stimulation pattern; these are the estimated peak EMF values during the 500 ns pulse duration. The time-averaged EMF values are much lower when considering the duty cycle, and would be less than 5% of these values.

The following is an example of a eukaryotic cell responding to the presence and absence of a pharmaceutical compound, DEX. The release of the drug from the nano-complex is controlled by the presence and absence of EMF from an EMF generating device. Two groups of toxin challenged murine neonatal microglial cells (BV-2) were used to evaluate reactive oxygen species (ROS) production and to determine whether such damaged cells could be rescued via EMF associated drug release. In one group, the challenge was bacterial derived LPS and in the other, the cells were directly insulted with hydrogen peroxide. BV-2 cells treated with Escherichia coli produced lipopolysaccharides (LPS) released pro-inflammatory cytokines that are induced by ROS via redox-dependent signaling pathways. CM-H2DCFDA is one such indicator compound for ROS and was used as a metric for ROS production. LPS (1 ug/ml) treated cells exhibited bright green fluorescence in the cytoplasm, marking the production of significant oxidative stress. The addition of DEX (1 ug/ml) to these LPS induced microglia is known to effectively suppressed ROS production during the inflammatory cascade. DEX coupled Ppy and gold nanoparticle vertically aligned rods also suppressed inflammation byproducts during EMF stimulation. A weaker inhibition of ROS activity occurred when short stimulation times were applied. Similarly, exposure to 20 ug/ml hydrogen peroxide resulted in more ROS byproducts in BV-2 cells. This enhanced production of ROS was reduced either by direct application of 10 ug/ml DEX or with 15 minutes of EMF stimulation to the DEX coupled Ppy and gold nanoparticle vertically aligned rods. These findings were further corroborated by the results of induced nitric oxide synthase (iNOS) measurements. Microglial cells were stained intensely with a Cy3 (red) fluorescence signal when exposed to LPS in a dose-dependent manner. iNOS expression in BV-2 cells after LPS challenge (without stimulation) also showed positive Cy3 staining, which is in agreement with the ROS results. In contrast, EMF stimulation of DEX coupled Ppy and goldnanoparticle vertically aligned rods resulted in a strong suppression of iNOS, indicating a reduced level of nitric oxide.

### FURTHER EXAMPLES

The following are further examples showing implementation and test results using the above described system and method.

We performed a lower-thoracic Spinal Cord Injury (SCI) in transgenic mice who produce a bioluminescent GFAP (Glial Acidic Fibrillary Protein: a specific product of “activated” glia, a major component of the forming scar after spinal cord injury). These mice permit bioluminescent imaging of the whole animal to define specific locations and intensities of GFAP production or its disappearance. The experimental therapy was to insert a small patch (˜1-2 mm²) of a gold base supporting a dense mat of vertically arranged Polypyrole nanowires (200 nm diameter×1800 nm). This “patch” was manually placed into the open SCI lesion at the time of surgery—attached to the surface of a single drop of sterile physiological saline by surface tension. The nanowire polymers were electromagnetically sensitive and doped during fabrication with Dexamethasone (DEX).

One SCI Control group (5 animals) did not receive any implant whatsoever.

A second SCI Control group (5 mice) possessed this Nanowire patch, placed into the SCI in an identical manner to experimental animals but they were not exposed to an Electromagnetic Field.

In Experimental mice, localized EMF exposure was limited to one period of 2 hours/day for up to a week at the local site of the SCI. The applied EMF of ˜40 gauss is required to induce the release of DEX from the PPyNWs.

GFAP—Luciferin (Luc) Transgenic mice (˜24 grams) were chosen as the SCI model because they are genetically engineered to produce a bioluminescent GFAP protein when it is expressed by reactive Astrocytes. This permits multi-dimensional imaging of GFAP, and its changing dynamics, in the living animal. Mice were deeply anesthetized with an injection of Ketamine/Xyzalzine and a standard dorsal, bilateral, hemilaminectomy procedure was performed to expose the lower thoracic spinal cord. Subsequently the cord was lightly compressed to approximately ¼th of its diameter using watchmaker's forceps under stereoscopic illumination. Our decision to lightly injure the cord was based on the desire to escape significant mortality in these transgenic animals, and that the aim of the surgery was to produce a measurable inflammation in the cord. The DEX—doped PPyNW attached to its gold base was lifted on a drop of sterile saline and deposited onto the exposed dorsal region of the compressed spinal cord. The lesion was then surgically closed in layers, and the animals were moved to their individual pens—and warmed with incandescent lights until they recovered. Further details on surgery and anesthesia can be found in online support materials.

Within three/four hours of surgery, awakened alert Experimental animals were lightly sedated by isoflurane to be quieted for their exposure to the EMF. All animals were treated similarly for their daily periods of bioluminescent imaging. The day following surgery, Experimental animals received their second EMF exposure period—and all animals were imaged at the Purdue Bioluminescent Imaging Laboratory (PBIL) by the end of that day.

In the active (Experimental) treatment, a magnetic coil was brought within a few mms of the surface of the suture-line. This delivered approximately 40 Gauss at an asymmetrical frequency at the level of the injury and imbedded PPyNW. Each day after the first treatment, this regimen was continued for 7 days. Only one animal of the 15 died, and was replaced, prior to the end of the experiment.

All Control and Experimental mice were taken to the PBIL. In this facility, each animal received an intraperitoneal injection of a luciferin solution and underwent imaging during a short period of immobilization with isoflurane. Bioluminescent imaging cannot provide raw data revealing; 1) the actual concentration of GFAP or, 2) astrocyte cell density. It can reveal: 1) highly specific localization of GFAP expression in the living animal, and 2) the relative GFAP intensity between these animals via photon radiation of the GFAP signature permitting quantification and statistical analysis.

There was a trend for the intensity of GFAP to become reduced in both groups of Control Animals. This steady reduction was expected from the biology, coincident with the maturation of reactive astrocytes which remained localized to the SCI. However, a statistical difference between the GFAP signals during this decline in the two Control Groups was not detected.

With regard to Astrocyte maturation and GFAP expression, there was: 1. always, and 2. in every Control animal, 3. and at every time point; a clear, unmistakable, and measurable GFAP signal was documented.

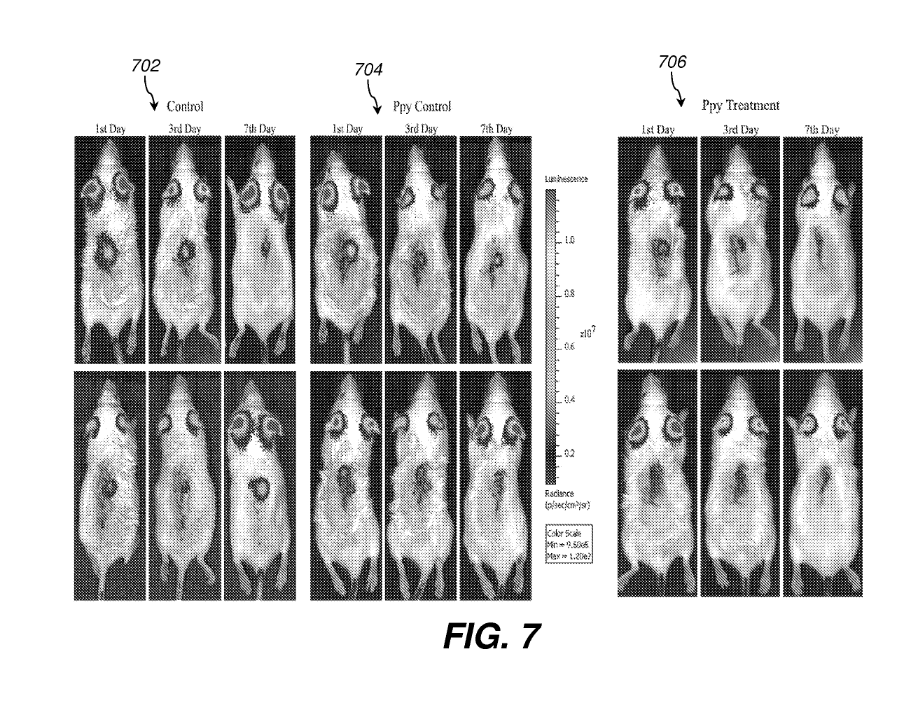

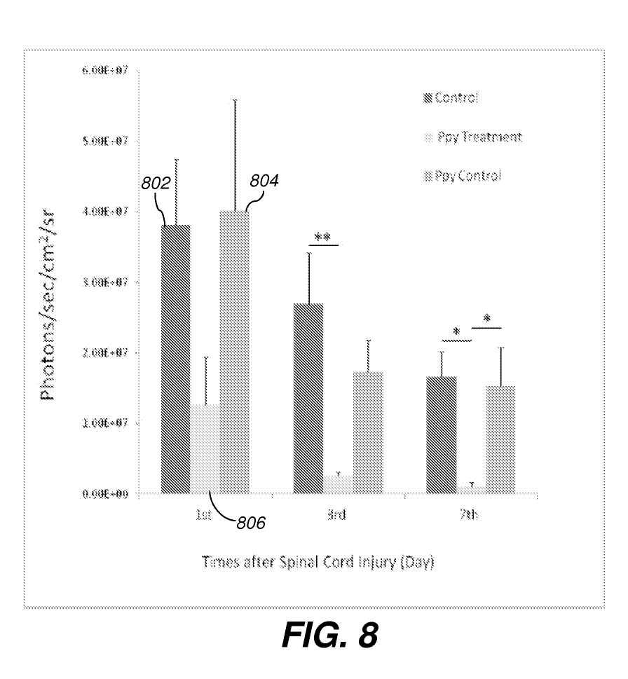

Importantly, a strong GFAP signal was also associated with ear infections in every animal of this transgenic breed. FIG. 7 shows typical untreated control (702), sham-treated Control Animals (704), as well as Experimental treated animals (706) for comparison. FIG. 8 also quantitates these data by comparing relative intensity (given in Photons/sec/cm²) of the GFAP in the lesion for untreated control animals (802), sham-treated control animals (804), and experimental treated animals (806).

In every case, the EMF—induced release of DEX produced a very steep decline in the GFAP signature—especially apparent by day 3. At the first measurement day post-injury, GFAP expression was already 60% weaker than Control values, though this difference did not reach significance given the wide variability of the means 24 hours after surgical injury (P≦0.05; Students T test). When assumptions concerning statistical normality was ignored, a very strong trend toward significance was observed on this first day (P=0.056). By day 3, the GFAP signal in Experimental animals compared to Controls was very significant—both visually and statistically. The standard error of the means (SEM) of the Experimental group compared to all Control samples was strikingly greater (P <0.001; Student's T Test). At post-surgery day 7; 2 of the 5 EMF—treated animals did not possess even a trace of the GFAP signal. The other animals in this group revealed GFAP expression, but at the barest level of detection. FIGS. 7 and 8 provides these comparisons.

It is important to point out that GFAP positive ear infections in all animals in this study provided a significant and telling internal control. This Infection was not reduced or otherwise affected in either Control or Experimental samples. We suggest the latter is explained by the likelihood that concentrations of DEX, released into the circulation from the localized Nanosource in the spinal cord after EMF exposure, would be virtually undetectable and of no significance to the ear infections. This observation provides formal proof that the local release of DEX by smart nanowires in comparatively high concentrations was restricted to the Spinal Cord lesion, where DEX-doped PPyNWs were surgically placed and later exposed to a directed EMF.

In conclusion, the PpyNW-based drug delivery system utilizes targeting by physical placement, and is not encumbered by side effects and other unknowns associated with immunological—based targeting and other modern techniques. The PpyNWs are simply placed where they are to be effective. This can be as simple as placement using a biopsy needle.

This scheme rules out its use in systemic disease—but is likely to be indicated, and extremely effective, in killing early tumor formations in breast, lung, prostrate, etc., where Clinical imaging indeed reveals these early circumscribed/localized neoplasms. It is called for in treatment of localized CNS injury—including local regions of ischemia in Stroke, as well as many localized and resistant infections and ulcerations in critical organs and tissues. The therapeutic chemical of choice to be delivered would be chosen based on the clinical problem: For example, BDNF (and other neuronal growth-promoting cytokines), Methylprednisilone, or DEX might be used in clinical SCI or Brain Trauma; specific and dangerous Chemotoxins for specific neoplasms; special anti-inflammatory and bactericides for severe localized infection in chronic bone non-unions or Decubitus ulcerations. Basically this procedure opens the door to many known therapies that cannot be easily or safely applied systemically—but till now could not be delivered continuously for many days to specific localized tissues.

The remote control of delivery is by a simple and reliable method. The EMF required to induce drug delivery, and to stop it, is completely non-invasive to the body, and of a character to be harmless.

The method of nanowire preparation used here is available for any therapeutic drug, chemotoxin, cytokine, bactericide, etc. as these agents are initially dissolved into the Ppy monomer solution prior to its polymerization.

Therefore, as revealed here, the tissue level response to a chosen clinically relevant chemical released into its local environment can be dramatic and sustained.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

1. An implantable time-release delivery system comprising;
at least one nano-complex of a plurality of vertically aligned rods fixed at one end to a substrate and configured to be implantable within a body, the plurality of vertically aligned rods comprising a conducting polymer, gold nanoparticles, and a compound; and
an electromagnetic field generating device configured to generate an electromagnetic field, positioned in a near field arrangement with respect to the nano-complex, the electromagnetic field causes release of the compound from the nano-complex into the body.

2. The system of claim 1, wherein the conducting polymer is polypyrrole.

3. The system of claim 1, wherein the compound is a pharmaceutical compound.

4. The system of claim 1, wherein the substrate comprises gold.

5. The system of claim 1, comprising:
a plurality of said nano-complexes, the substrates of said nano-complexes coupled together.

6. The system of claim 5, wherein said substrates are coupled together by an attachment edge of said substrates.

7. The system of claim 1, wherein said rods have a thickness between 20 nm and 2000 nm.

8. The system of claim 1, wherein the electromagnetic field generating device is configured to produce EMF pulses, the pulses having a duration between 200 and 700 nS.

9. The system of claim 1, wherein the electronmagnetic field generating device is configured to produce a plurality of positive EMF pulses, followed by at least one negative EMF pulse.

10. (canceled)

11. A drug delivery device comprising;
a nano-complex of a plurality of vertically aligned rods fixed at one end to a substrate and configured to respond to an electromagnetic field, the plurality of vertically aligned rods comprising polypyrrole, gold, and a compound.

12. A method of controlling compound release comprising;
providing at least one nano-complex of a plurality of vertically aligned rods fixed at one end to a substrate and configured to respond to an electromagnetic field, the plurality of vertically aligned rods comprising a conducting polymer, gold, and at least one compound;
providing at least one pulse of electromagnetic energy to the nano-complex in a range of 20 to 40 G, to thereby release the at least one compound from the nano-complex; and
stopping the pulse of electromagnetic energy to thereby stop the release of the at least one compound.

13. The method of claim 12, wherein the conducting polymer is polypyrrole.

14. The method of claim 12, wherein the compound is a pharmaceutical compound.

15. The method of claim 12, wherein the substrate comprises gold.

16. The method of claim 12, wherein said at least one nano-complex comprises a plurality of said nano-complexes, the substrates of said nano-complexes coupled together.

17. The method of claim 16, wherein said substrates are coupled together by an attachment edge of said substrates.

18. The method of claim 12, wherein said rods have a thickness between 20 nm and 2000 nm.

19. The method of claim 12, wherein said pulse has a duration between 200 and 700 nS.

20. The system of claim 12, wherein said providing at least one pulse comprises providing a plurality of positive pulses, followed by at least one negative pulse.
